(19) United States
(12) Patent Application Publication
(10) Pub. No.: US 2010/0227836 A1
(43) Pub. Date: Sep. 9, 2010

(54) PARENTERAL ADMINISTRATION OF A GLUCOSAMINE

(75) Inventors: JENNIFER H. ELISSEEFF, BALTIMORE, MD (US); SHYNI VARGHESE, SAN DIEGO, CA (US); JEANNINE COBURN, BALTIMORE, MD (US); MATTHEW GIBSON, BALTIMORE, MD (US); ZAYNA NAHAS, MIAMI, FL (US); ZHAOYANG YE, BALTIMORE, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, BALTIMORE, MD (US)

(21) Appl. No.: 12/571,367

(22) Filed: Sep. 30, 2009

Related U.S. Application Data
(63) Continuation of application No. PCT/US09/51573, filed on Jul. 23, 2009.
(60) Provisional application No. 61/135,763, filed on Jul. 23, 2008.

(51) Int. Cl. A61K 31/7008; C07H 5/06; A61K 31/728; A61K 31/737

(52) U.S. Cl. 514/54; 536/55.2; 536/54; 536/55; 514/62

(57) ABSTRACT

Materials and methods for local delivery of a glucosamine are provided to facilitate bone and cartilage growth.

### CROSS-REFERENCE TO RELATED APPLICATION

This application claims the benefit of copending application U.S. Provisional Application Ser. No. 61/135,763 filed on Jul. 23, 2008, the entire contents of which are incorporated herein by reference.

### BACKGROUND OF INVENTION

Glucosamine (GlcN) is a popular orally administered nutraceutical taken to improve joint health. Whether and how such glucosamine has a tangible biological effect is unclear. Very low levels, on the order of 6 μM in serum and 0.5 μM in synovial fluid were observed following nasogastric intake of 20 mg/kg/day of GlcN (Laverty et al., Arth Rheum 52:181-191, 2005).

Kulkarni (U.S. Pat. No. 6,822,064) teach polymerized macromers containing, for example, N-acetylglucosamine (NAG). The molecules of interest comprise a backbone molecule to which NAG, among many other sugars, is covalently bound. The macromers are to bind to and inactivate lysozyme. The macromers are stable and resistant to degradation, an advantage over natural polymers.

The instant invention is premised on the observation that GlcN has a beneficial effect on bone and joint health when present in particular local concentrations. In view thereof, the instant invention is directed to materials and methods to achieve that local effective concentration of bioavailable glucosamine.

### SUMMARY OF THE INVENTION

It is an object of the invention to provide compositions of various forms for use as devices and vehicles in a body to provide for an effective local concentration of glucosamine. The compositions can be in liquid or solid form. Thus, an object of the invention is to provide, for example, a replacement synovial fluid; a scaffold for tissue engineering; a film for wound healing, shaping or for lining or coating a surface; forms for drug delivery, such as microcapsules, fibers and so on, that comprise a glucosamine, which provide for the desired local concentration of therapeutic glucosamine.

It is another object of the instant invention to provide for the local administration of glucosamine. The devices of interest enable biologically sufficient concentrations of therapeutic glucosamine at a body site in need of treatment.

Those and other objects have been attained in the development of various devices comprising glucosamine, such as a functionalized glucosamine or administrable glucosamine preparations, and the use thereof to attain therapeutic levels of glucosamine at a site in need thereof. Suitable other biomolecules can be conjugated with GlcN including, for example, hyaluronic acid and chondroitin sulfate, or can be administered concurrently or sequentially with GlcN.

### DETAILED DESCRIPTION OF THE INVENTION

The invention relates to products and methods for treating, for example, joint ailments where replacement or supplementation of glucosamine is desired.

Glucosamine (GlcN) is meant to indicate the amino sugar compound carrying four hydroxyl groups and an amine group of formula C₆H₁₃NO₅. However, for the purposes of the invention, glucosamine derivatives, analogs and the like are included in the definition of a glucosamine. Thus, for example, NAG is meant to be included in the term glucosamine.

In one embodiment, a molecule of interest is contained within a microcapsule, microsphere and so on, which terms are used synonymously. Hence, a glucosamine can be contained within a microcapsule. The microcapsules can be made using standard reagents and chemistries. As known in the art, the outer surface of a microcapsule can be designed to carry certain properties or molecules, for example. Such surface modifications enable targeting of microspheres, adherence of same and so on.

In another embodiment, a molecule of interest comprises a fiber, a microfiber, a nanofiber, a fibril and so on, which terms are used synonymously. The nanofiber can be made using standard materials and chemistries, and thus, can comprise, for example, an outer shell composed of a biodegradable material, such as a biodegradable polyester or chitosan, and contained within the core portion of the fiber is a glucosamine. In other embodiments, a glucosamine is doped into the component(s) comprising the nanofiber. In other embodiments, the nanofiber can be coated with a molecule providing for a desired characteristic. Hence, a fiber can be coated with a polar molecule for targeting purposes, adhering purposes, retaining purposes to obtain a delayed release of materials contained within and in a nanofiber of interest and so on. An example of such a polar molecule is chondroitin sulfate.

A glucosamine of interest generally is included with a vehicle. That vehicle can be a physiologically acceptable carrier, excipient or diluent, such as a saline, water, a buffer and so on, as known in the pharmaceutic arts. The vehicle also can be a molecule which carriers, transports, stores and so on GlcN, which vehicle can be composed of, for example, a biologically compatible polymer. Thus, in some embodiments, a biologically compatible polymer can serve as a carrier of a glucosamine.

The term “biologically compatible polymer” or “biocompatible” refers to a naturally occurring polymer or one that is not toxic to the host. Generally, the metabolites of a device of interest also are not toxic to the host. It is not necessary that any subject composition have a purity of 100% to be deemed biocompatible; indeed, it is only necessary that the subject compositions be non-toxic to the host. Hence, a subject composition may comprise monomer, polymers or portions thereof comprising 99%, 98%, 97%, 96%, 95%, 90%, 85%, 80%, 75% or even less of biocompatible monomer, polymers or portions thereof, e.g., including monomers, polymers or portions thereof, and other materials and excipients described herein, and still be biocompatible.

To determine whether a polymer or other material is biocompatible, it may be necessary to conduct a toxicity analysis. Such assays are well known in the art. One example of such an assay may be performed with, for example, live carcinoma cells in the following manner: a sample of the intact molecule or a sample wherein the molecule is degraded in IM NaOH at 37° C. until complete degradation is observed is used. The solution is then neutralized with IM HCl. About 200 pL of various concentrations of the sample are placed in 96-well tissue culture plates and seeded with human carcinoma cells at about 10⁴/well density. The samples are incubated with the cells for 48 hours. The results of the assay may be plotted as % relative growth vs. concentration of sample in the tissue culture well. In addition, monomers, polymers, polymer structures and formulations of the present invention may also be evaluated by well-known in vivo tests, such as subcutaneous implantation in rats to confirm that they do not cause significant levels of irritation or inflammation at the subcutaneous implantation sites. Acceptable levels of toxicity are as known in the art.

A GlcN molecule of interest is one that is not toxic to a host, such as a mammal, Determining safety in a host is a well known exercise in the food and drug arts, and includes, for example, in vitro studies on cells and tissues, and perhaps, organs, animal studies and early human clinical trials as described herein.

In many of the devices of interest, a biological molecule, a backbone molecule, a glucosamine and so on are joined or attached by biologically labile linkages. Thus, the devices of interest can be or comprise biodegradable portions.

By biodegradable is meant that the polymer or particular bonds of the polymer are cleaved under normal physiological processes in a mammal. Generally, the polymer degradation products are non-toxic or biocompatible as well.

The term “biodegradable” is art-recognized and is intended to indicate that an object degrades during use. In general, degradation attributable to biodegradability involves the degradation of a biodegradable polymer into oligomers or its component subunits, or digestion, e.g., by a biochemical process, of the polymer into smaller subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain to the polymer backbone. The side chain is one that can contain a GlcN. Alternatively, a therapeutic agent, biologically active agent or other chemical moiety attached as a side chain to a polyGlcN may be released by biodegradation. In certain embodiments, one or the other or both general types of biodegradation may occur during use of a polymer of interest. As used herein, the term “biodegradation” encompasses both general types of biodegradation as the overall desired function of the functionalized polymer of interest wanes.

The degradation rate of a biodegradable polymer often depends in part on a variety of factors, including the chemical identity of linkages; the molecular weight, crystallinity, biostability and degree of cross-linking of such polymer; the physical characteristics of the implant, such as the shape and size; the mode and location of administration; and so on. For example, the greater the molecular weight, the higher the degree of crystallinity, and/or the greater the biostability, the biodegradation of any biodegradable polymer is usually slower. The term “biodegradable” is intended to cover materials and processes also termed “bioerodible”. Generally, the rate of degradation is a design choice based on the monomers, functional groups, added ingredients and the like that are used.

In certain embodiments, the biodegradation rate of such polymer may be characterized by the presence of enzymes, for example, a particular protease, lipase, saccharidase and so on. In such circumstances, the biodegradation rate may depend on not only the chemical identity and physical characteristics of the polymer matrix, but also on the identity, use, presence and the like of any such enzyme.

Thus, a GlcN molecule of interest can be one that carries plural GlcN residues and releases GlcN residues thereby making the GlcN molecule bioavailable, but also can be one that releases monomers or oligomers of the backbone molecule.

GlcN is a naturally occurring molecule and has nutritive and effector functions. GlcN, for example, is compatible with and promotes stem cell growth and differentiation, for example, of mesenchymal stem cells to form chondrocytes. GlcN can have a role in tissue development and repair, such as cartilage growth and development, in general. See, for example, Varghese et al. OsteoArthritis and Cartilage 15, 59, 2007. There it was observed that particular concentrations, a narrow window of no more than about 2 mM of glucosamine, had a beneficial effect on cell growth, matrix production and gene expression. Thus, for example, GlcN upregulated transforming growth factor β1 (TGF-β1) expression. That growth factor may have a role in stimulating extracellular matrix (ECM) components.

Therefore, placing a GlcN composition of interest into a bone or cartilage defect, whether arising by normal wear and tear, from an injury or purposely to stimulate repair, such as by microfracture, can serve not only a structural or mechanical role by filling the defect and providing structural support, but also a nutritive role by stimulating cell growth, by stimulating stem cell differentiation, and by stimulating bone and cartilage growth. A composition that provides about a 2 mM local concentration of GlcN is beneficial. Other concentrations, such as 1-3 mM can be advantageous, however, amounts much greater than 2 mM may not be advantageous or cost effective. Thus, a suitable local concentration can be about 3 mM, about 2.9 mM, about 2.8 mM, about 2.7 mM, about 2.6 mM, about 2.5 mM, about 2.4 mM, about 2.3 mM, about 2.2 mM, about 2.1 mM, about 2.0 mM, about 1.9 mM, about 1.8 mM, about 1.7 mM, about 1.6 mM, about 1.5 mM, about 1.4 mM, about 1.3 mM, about 1.2 mM, about 1.1 mM, about 1.0 mM, about 0.9 mM, about 0.8 mM, about 0.7 mM, about 0.6 mM, about 0.5 mM or so on. An artisan can determine a suitable local concentration of GlcN practicing methods known in the pharmaceutic arts, and that determination will govern the nature and composition of the GlcN composition of interest to obtain the desired concentration of GlcN.

All of the reagents necessary to make a composition of interest are commercially available or can be attained from natural sources.

Delivery of a glucosamine of interest can be by any means to obtain the local therapeutic concentration of GlcN as disclosed herein. Lower levels can be less effective, and at higher levels, GlcN can have a detrimental effect.

Hence, to obtain rapid impact, a glucosamine can be applied to a site in need of treatment using a suitable delivery means so that the effective concentration of glucosamine desired is obtained as soon as possible. To obtain a more sustained local effective concentration of glucosamine, other delivery means can be used, including using different forms, derivatives, analogs and the like of glucosamine, such as polymerized forms of glucosamine and polymers carrying plural glucosamine residues, depots, sustained release pharmaceutical formulations, such as microcapsules, emulsions, coated microcapsules and so on, as known in the art, and so on. A combination of rapid and sustained release delivery means can be used to provide for the effective local concentration of glucosamine for a desired period of time.

Thus, a glucosamine can be delivered locally by injection or instillation of a glucosamine, for example, into a joint. The glucosamine can be any form of glucosamine. For example, glucosamine per se, can be prepared into a liquid, injectable form for direct administration to a site in need of treatment. The liquid carrier, excipient or diluent can be any one that is pharmaceutically acceptable, as known in the art, such as, sterile water, a saline, a buffer, and so on. The liquid can contain other excipients, as known in the art to obtain desired characteristics, such as preservatives, buffers, thickeners and so on, as known in the art. Reference can be made to Remington: The Science and Practice of Pharmacy.

The formulation or preparation can be a solution, emulsion and so on comprising a glucosamine for rapid and/or delayed release. The liquid can comprise one or more components that will cause for a delayed degradation of said liquid to obtain a tonic and delayed release of glucosamine from said liquid. Thus, the solution can be, for example, an oil and water emulsion, such as an adjuvant.

For example, in an experimental model of osteoarthritis in rodents, rat knees were surgically manipulated to obtain histological manifestations of osteoarthritis (Janusz et al., Osteoarth, Cart. 10:785-791, 2002). Four weeks after treatment, injured joints were untreated, or treated with a PBS control, a 2 mM glucosamine solution or a 1:1 mixture of 2 mM glucosamine solution and CSMA-aldehyde (chondroitin sulfate methacrylate) (Wang et al., Nat. Mater. 6:385-392, 2007). Treatments were transcutaneous, intraarticular injections weekly for three weeks. The animals were sacrificed and histologic preparations were made of the treated joints and stained for cartilage, using, for example, safranin-O. Florid cartilage development was observed in the joints treated with glucosamine and with the glucosamine mixture as compared to the PBS control and the untreated control joints. Cartilage was best developed and well organized in the glucosamine and glucosamine mixture treatment groups.

In another embodiment, the solution comprises microcapsules or microbeads comprising a glucosamine. The microcapsules can be constructed of materials that achieve a sustained and/or delayed release profile, as known in the art.

In yet another embodiment, a device of interest comprises a scaffold material to provide structural support to promote, for example, cell growth, ECM production and so on. The scaffold can be made from any biocompatible material, such as a GlcN, or can be made to contain a GlcN, either as part of the structure per se, or entrapped, contained, carried and so on by the scaffold structure. The scaffold per se can be biodegradable to release GlcN at a controlled rate, or can be configured or composed to release GlcN contained therein at a controlled rate, to achieve the local concentration of GlcN of interest.

Hence, in certain embodiments, novel mechanisms for polymerizing glucosamine are described. For example reaction of GlcN with phosgene produces a polyglucosaminocarbamate via an isocyanate intermediate. Such a polymer is useful as a biocompatible and biodegradable polymer as the carbamate functions can be hydrolyzed under physiological conditions to yield glucosamine monomers.

To facilitate reaction to obtain a device of interest, a GlcN of interest, or other reactant of interest, such as a component of a microcapsule, a nanofiber, a scaffold and so on can be derivatizecl to contain a reactive substitution, such as an alcohol group, an ester group and so on.

Thus, a GlcN molecule of interest, whether a monomer or part of a polymer, may be functionalized to contain reactive groups. That enables a GlcN molecule of interest to be covalently attached to a matrix, tissue and the like, enables a GlcN to be polymerized or enables a GlcN molecule of interest to contribute to a biomaterial. Thus a functionalized GlcN molecule of interest can react with a functionalized polymer or functionalized hydrogel, for example.

A reactive moiety includes any moiety that reacts with a suitable element, chemical group or chemical site on a target entity. One set of target entities are biological structures, such as cells, tissues, organs and the like. A functional group on the biologically compatible polymer reactive with a biological surface moiety includes any functional group that reacts with a suitable element, chemical group or chemical site on a surface of a biological structure, such as a cell, tissue, organ and the like. Thus, a suitable element, chemical group or chemical site on the surface of a biological structure would be a reactive group found in, for example, a carbohydrate, an amino acid or a nucleic acid, such as an amine group, a carboxylic acid group, a hydroxyl group, a sulfate group and so on. Accordingly, a suitable reactive moiety would be one that reacts with an amine group, a hydroxyl group and so on of the surface of a biological structure. A suitable functional group would be one that reacts with an amine group, a hydroxyl group and so on of the surface of a biological structure. Another example is an aldehyde group. Those functional groups also enable reaction of suitable reactants with self or with other reactants.

Other reactive moieties are those which react with elements, chemical groups or chemical sites on biologically compatible materials, such as implants, tissues, prostheses, other devices and the like.

Other functional groups on the biologically compatible polymer are those which react with elements, chemical groups or chemical sites on a bridging molecule.

A reactive moiety or functional group (which terms, for the purposes of the invention, are considered equivalent) may include alkenyl moieties such as acrylates, methacrylates, dimethacrylates, oligoacrylates, oligomethacrylates, ethacrylates, itaconates or acrylamides. Further reactive moieties include carboxylates and aldehydes. Other reactive moieties may include ethylenically unsaturated monomers including, for example, alkyl esters of acrylic or methacrylic acid such as methyl methacrylate, ethyl methacrylate, butyl methacrylate, ethyl acrylate, butyl acrylate, hexyl acrylate, n-octyl acrylate, lauryl methacrylate, 2-ethylhexyl-methacrylate, nonyl acrylate, benzyl methacrylate, the hydroxyalkyl esters of the same acids such as 2-hydroxyethyl acrylate, 2-hydroxyethyl methacrylate, and 2-hydroxypropyl methacrylate, the nitrile and amides of the same acids such as acrylonitrile, methacrylonitrile, methacrylamide, vinyl acetate, vinyl propionate, vinylidene chloride, vinyl chloride, and vinyl aromatic compounds such as styrene, t-butyl styrene and vinyl toluene, dialkyl maleates, dialkyl itaconates, dialkyl methylene malonates, isoprene and butadiene. Suitable ethylenically unsaturated monomers containing carboxylic acid groups include acrylic monomers such as acrylic acid, methacrylic acid, ethacrylic acid, itaconic acid, maleic acid, fumaric acid, monoalkyl itaconate including monomethyl itaconate, monoethyl itaconate, and monobutyl itaconate, monoalkyl maleate including monomethyl maleate, monoethyl maleate, and monobutyl maleate, citraconic acid and styrene carboxylic acid. Suitable polyethylenically unsaturated monomers include butadiene, isoprene, allylmethacrylate, diacrylates of alkyl diols such as butanediol diacrylate and hexanediol diacrylate, divinyl benzene and the like.

In some embodiments, a monomer of a biologically compatible polymer may be functionalized through one or more thio, carboxylic acid or alcohol moiety located on a monomer of the biopolymer.

The reactive moieties or functional groups are attached to the monomer or biologically compatible polymer using known chemistries based on design choice. Thus, in producing, for example, a functionalized saccharide, a solution comprising the saccharide and a first functional group reactant, such as an alkylene or an acrylate group, can be mixed, The solution is stirred, for example, for at least 10 days, at least 11 days or at least 15 days. Alternatively, the solution may be stirred or maintained for about 10 to about 15 days or about 14 to about 15 days. The solution may include a polar solvent, for example an aqueous solvent.

For example, methacrylic anhydride, methacryloyl chloride and glycidyl methacrylate may be used to add methacrylate groups to one or more monomers of a polymer chain. Glycidyl methacrylate may be used, for example, for efficiency of reaction. Further, the modification reagents may be chosen to optimize for a lack of cytotoxic byproducts.

A suitable method for making a polymer with aldehyde groups is to treat a molecule with adjacent hydroxyl groups with a periodate salt, as known in the art, to yield for example, a chondroitin sulfate that is functionalized with a methacrylate group as used herein.

The term “functionalized” refers to a modification of an existing molecular entity, structure or site to generate or to introduce a new reactive or more reactive group, such as an acetyl group or a group (e.g., acrylate group) that is capable of undergoing reaction with another functional group (e.g., a sulfhydryl group) to form, for example, a covalent bond. For example, carboxylic acid groups can be functionalized by reaction with an acyl halide, e.g., an acyl chloride, again, using known procedures, to provide a new reactive functional group in the form of an anhydride.

A number of varying reactive moieties can be used in the practice of the instant invention. The following provides a non-exhaustive but an exemplified listing of substitutional groups.

The term “aliphatic” is an art-recognized term and includes linear, branched and cyclic alkanes, alkenes or alkynes. In certain embodiments, aliphatic groups in the present invention are linear or branched and have from 1 to about 20 carbon atoms, or more.

The term “alkyl” is art-recognized and includes saturated aliphatic groups, including straight-chain alkyl groups, branched-chain alkyl groups, cycloalkyl (alicyclic) groups, alkyl substituted cycloalkyl groups and cycloalkyl substituted alkyl groups. In certain embodiments, a straight chain or branched chain alkyl has about 30 or fewer carbon atoms in its backbone (e.g., C₁-C₃₀ for straight chain and C₃-C₃₀ for branched chain), and alternatively, about 20 or fewer carbon atoms. Likewise, cycloalkyls have from about 3 to about 10 carbon atoms in their ring structure, and alternatively, about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen atom on one or more carbons of the hydrocarbon backbone. Such substituents may include, for example, a halogen, a hydroxyl, a carbonyl (such as a carboxyl, an alkoxycarbonyl, a formyl or an acyl), a thiocarbonyl (such as a thioester, a thioacetate or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the-art that-the-moieties substituted on the hydrocarbon chain may themselves be substituted, if appropriate. For instance, the substituents of a substituted alkyl may include substituted and unsubstituted forms of amino, azido, amino, amido, phosphoryl (including phosphonate and phosphinate), sulfonyl (including sulfate, sulfonamido, sulfamoyl and sulfonate) and silyl groups, as well as ethers, alkylthios, carbonyls (including ketones, aldehydes, carboxylates and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN and the like.

The term “aralkyl” is art-recognized and includes aryl groups (e.g., an aromatic or heteroaromatic group).

The terms “alkenyl” and “alkynyl” are art-recognized and include unsaturated aliphatic groups analogous in length and possible substitution of the alkyls described above, but that contain at least one double or triple bond, respectively.

Unless the number of carbons is otherwise specified, “lower alkyl” refers to an alkyl group, as defined above, but having from one to ten carbons, alternatively, from one to about six carbon atoms in the backbone structure. Likewise, “lower alkenyl” and “lower alkynyl” have similar chain lengths.

A “methacrylate” refers to a vinylic carboxylate, for example, a methacrylic acid in which the acidic hydrogen has been replaced. Representative methacrylic acids include acrylic, methacrylic, chloroacrylic, cyano acrylic, ethylacrylic, maleic, fumaric, itaconic and half esters of the latter dicarboxylic acids.

The term “heteroatom” is art-recognized and in an organic molecule, generally includes an atom of any element other than carbon or hydrogen. Illustrative heteroatoms include boron, nitrogen, oxygen, phosphorus, sulfur and selenium.

The term “aryl” is art-recognized and includes, for example, 5-membered, 6-membered and 7-membered single ring aromatic groups that may include from zero to four heteroatoms, for example, benzene, pyrrole, furan, thiophene, imidazole, oxazole, thiazole, triazole, pyrazole, pyridine, pyrazine, pyridazine, pyrimidine and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulthydryl, imino, amido, phosphonate, phosphinate, carbonyl, carboxyl, silyl, ether, alkylthio, sulfonyl, sulfonamido, ketone, aldehyde, ester, heterocyclyl, aromatic or hetero aromatic moieties, —CF₃, —CN or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls, or rings joined by non-cyclic moieties.

The terms “ortho”, “meta” and “para” are art-recognized and apply, for example, to 1,2-disubstituted, 1,3-disubstituted and 1,4-disubstituted benzenes, respectively. For example, the names 1,2-dimethylbenzene and ortho-dimethylbenzene are synonymous.

The terms “heterocyclyl” and “heterocyclic group” are art-recognized and include 3-membered to about 10-membered ring structures, such as 3-membered to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams, such as azetidinones and pyrrolidinones, sultams, sultones and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN or the like.

The terms “polycyclyl” and “polycyclic group” are art-recognized and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN or the like.

The following art-recognized terms have the following meanings: “nitro” means —NO₂; the term “halogen” designates —F, —Cl, —Br or —I; the term “sulfhydryl” means —SH; the term “hydroxyl” or “hydroxy” means —OH; and the term “sulfonyl” means —SO₂—.

The terms “amine” and “amino” are art-recognized and include both unsubstituted and substituted amines, as well as primary, secondary tertiary amines, which may be functionalized. The term “alkylamine” includes an amine group, as defined above, having a substituted or unsubstituted alkyl attached thereto. The term “acylamino” is art-recognized and includes a amine having a substituted or unsubstituted acyl group attached thereto. The term “amido” is art-recognized as an amino-substituted carbonyl.

Certain monomeric subunits of the present invention may exist in particular geometric or stereoisomeric forms. In addition, polymers and other compositions of the present invention may also be optically active. The present invention contemplates all such compounds, including cis and trans isomers, R and S enantiomers, diastereomers, d isomers, l isomers, the racemic mixtures thereof, and other mixtures thereof, as falling within the scope of the invention. Additional asymmetric carbon atoms may be present in a substituent, such as an alkyl group. All such isomers, as well as mixtures thereof, are intended to be included in the instant invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino or an acidic functional group, such as carboxyl, diastereomeric salts are formed with-an-appropriate optically active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with the permitted valency of the substituted atom and the substituent, and that the substitution results in a stable compound, e.g., which does not spontaneously undergo transformation, such as by rearrangement, cyclization, elimination or other reaction.

The term “substituted” is also contemplated to include all permissible substituents of organic compounds. In a broad aspect, the permissible substituents include acyclic and cyclic, branched and unbranched, carbocyclic and heterocyclic, and aromatic and non-aromatic substituents of organic compounds. Illustrative substituents include, for example, those described hereinabove. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. The instant invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. See, for example, WO2006/089167.

Thus, for example, the 6-OH group of glucosamine can be functionalized by reacting GlcN with, for example Fmoc to protect the amine group, and then with, for example, acryloyl chloride, which reacts with the 6 hydroxy group. The Fmoc group then can be removed to yield a glucosamine carrying, in this example, an acryloyl group at the 6 position.

The 2-amine group also is amenable to substitution or functionalization. For example, glucosamine can be reacted with allyl chloroformate, wherein the latter attaches to the amine group with liberation of a mole of HCl. See also, WO 2006/127977.

In some embodiments, the number of the reactive moieties per polymeric unit may be at least one moiety per about 10 monomeric units, or at least about 2 moieties per about 10 monomeric units. Alternatively, the number of reactive moieties per polymeric unit may be at least one moiety per about 12 monomeric units, or per about 14 monomeric units. For example, there may be at least about one reactive moiety per 15 or more monomeric units. The number of moieties also can range from one per monomer, one per two monomers, one per three monomers, one per 4, 5, 6, 7, 8 or 9 monomers.

Also, a polymer of interest may contain plural species of reactive moieties to provide a directionality to the polymer. When, for example, a polymer contains two species of reactive moieties, the ratio of one of the two reactive moieties to the other can be 5:1, 9:2, 4:1, 7:2, 3:1, 5:2, 2:1, 3:2, 1:1, 2:3, 1:2, 2:5, 1 :3,2:7, 1:4,2:9 or 1:5 along the full length of the polymer. Preferably, each of the functional moieties is regularly distributed along the length of the polymer and in substantially equal molar amounts. However, the amount of any one reactive moiety type is optimized for reaction with the intended target entity and may result in amounts where the ratio of the two types of reactive moieties deviates from unity.

For example, polyethylene oxide-diacrylate (PEODA) may be used to form a hydrogel, and cross-linked polymer matrices may include cogels of an acrylated GlcN and PEODA. Alternatively, the gels can be constructed of polyethylene glycol diacrylate (PEGDA). GlcN can be incorporated into the gel formation reaction and the amine group of GlcN can be reacted with the vinyl group using UV light as an initiator, as known in the art. In an alternative format, the 6 hydroxyl group is made to react with the vinyl group to produce a labile ester bond, which are known to be hydrolysable under physiological conditions. The cogels formed thereby will have properties different from that of the two parent compounds, and properties of the cogel will vary based on the ratio of the two reactants. Examples of derivatized hydrogels can be found in WO 2004/029137.

In other embodiments, a GlcN is added to a molecular carrier, such as a polymer. That polymer can be biodegradable per se, that is, bonds forming the backbone can be degradable, or the bonds linking the GlcN residues to the carrier are degradable.

Thus, for example, a polyalcohol, a polyamine, a polylysine and so on can be reacted with GlcN and phosgene to yield a polyol GlcN, a polyamino GlcN or a polyLys GlcN, respectively. Under certain reaction conditions, the reaction involves isocyanate intermediates. Thus, when Lys is reacted with phosgene, lysine diisocyante is produced. When that is reacted with glucosamine, polylysinoglucosamino carbamate is produced.

In one embodiment, a GlcN of interest is encapsulated, encompasses, contained within, incorporated in, a component of and so on of a vehicle, such as a capsule, particle and so on (collectively identified as capsules), or a fiber and so on (collectively identified as fibers). Materials for making such capsules and fibers are known in the art, and include for example, polymers, such as celluloses, polyesters, polyacrylamides, polycaprolactones, polyvinylamines, polyvinylalcohols, polyglycolides, polylactides, chitosan, copolymers and so on. Also, materials known for making microcapsule and microparticle drug delivery forms, such as celluloses, methacrylates, other polysaccharides and so on, as known in the pharmaceutic arts can be used.

The mechanical properties of a polymer or a multi-layer polymer, such as a scaffold, may also be related to the pore structure. For applications in tissue engineering, scaffolds with different mechanical properties are produced depending on the desired clinical application. For example, scaffolds for cartilage tissue engineering in the articular joint must survive higher mechanical stresses than a cartilage tissue engineering system in other body sites. Thus, hydrogels with mechanical properties that are easily manipulated may be desired.

Cytotoxicity of the biopolymer scaffold system may be evaluated with any suitable cells, such as fibroblasts, by, for example, using a live-dead fluorescent cell assay and a suitable indicator of viability, such as a vital stain, such as a tetrazolium dye, such as MTT, a compound that actively metabolizing cells convert from yellow to purple.

A composition or device of interest can comprise other active agents. By active agent is meant an entity that elicits, causes, obtains and so on a pharmacologic, physiologic, biologic or some level of response in a host.

The terms “active agent,” “pharmaceutically active agent” and “biologically active agent” are used interchangeably herein to refer to a chemical or biological compound that induces a desired physical, pharmacological, biological or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. As described herein, a biologically active agent includes a living entity, such as a virus, microbe or cell.

The biologically active agent may vary widely with the intended purpose for the composition. The term “active” is art-recognized and refers to any chemical moiety that has a biological, physiological or pharmacological activity that acts locally or systemically in a subject. Examples of biologically active agents, that may be referred to as “drugs”, are described in well-known literature references such as the Merck Index, the Physicians Desk Reference and The Pharmacological Basis of Therapeutics, and include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released by the subject composition, for example, into adjacent tissues or fluids on administration to a subject.

Further examples of biologically active agents include, without limitation, enzymes, receptor antagonists or agonists, hormones, growth factors, autogenous bone marrow, antibiotics, antimicrobial agents and antibodies. The term “biologically active agent” is also intended to encompass various cell types and nucleic acids that can be incorporated into the compositions of the invention. Thus, a GlcN composition can contain collagen and other biological molecules. The GlcN composition can contain other molecules associated with cell tissue and biological adhesion in general, such as ROD peptides. The GlcN composition can comprise other biocompatible polymers, such as those associated with the bone, cartilage, ECM and so on. Hence, chondroitin sulfate, heparan sulfate, other glycosaminoglycans, aggrecans, proteoglycans, collagens, heparin, keratan sulfate, dermatan sulfate, hyaluronic acid, and other molecules associated with cartilage and bone, for example, can be included in a composition of interest. The biological agent can be obtained naturally, or derivatized and substituted as taught herein. The active ingredients can be combined in a single formulation or administered separately.

In certain embodiments, the subject compositions comprise about 1% to about 75% or more by weight of the total composition, alternatively about 2.5%, 5%, 10%, 20%, 30%, 40%, 50%, 60%, 70% or more, of a biologically active agent. Biologically active agents also include living entities, such as cells. Thus, for example, mesenchymal stem cells can be attached to or entrapped within a matrix comprising the polymers of interest. Mesenchymal stem cells may not be differentiated and therefore may differentiate to form various types of new cells due to the presence of an active agent, such as GlcN, or the effects (chemical, physical etc.) of the local tissue environment. Examples of mesenchymal stem cells include bone marrow cells, osteoblasts, chondrocytes and fibroblasts. For example, osteoblasts can be delivered to the site of a bone defect to produce new bone; chondrocytes can be delivered to the site of a cartilage defect to produce new cartilage; fibroblasts can be delivered to produce collagen wherever new connective tissue is needed; neurectodermal cells can be delivered to form new nerve tissue; epithelial cells can be delivered to form new epithelial tissues, such as liver, pancreas etc.

The cells may be either allogeneic or xenogeneic in origin. For example, the compositions can be used to deliver cells of other species that have been genetically modified, in some embodiments, the compositions of the invention may not easily be degraded in vivo, cells entrapped within the polymer compositions will be isolated from the patient cells and, as such, should not provoke an immune response when returned to the patient.

To entrap the cells within a polymer, the cells may, for example be mixed with a composition comprising functionalized polymer, and optionally, a further biocompatible polymer. That may occur through a particular reaction or may occur during the making of a multiple layer polymer. Alternatively, the cells may be contained within a target entity attached to a polymer of interest.

Various forms of the biologically active agents may be used. These include, without limitation, such forms as uncharged molecules, molecular complexes, salts, ethers, esters, amides, and the like, which are biologically activated when implanted, injected or otherwise placed into a subject.

In some embodiments, the invention is directed to articles of manufacture, such as, kits. In certain embodiments, this invention contemplates a kit including subject compositions and instructions for use. For example, the kit may comprise a GlcN biologically compatible polymer, a microcapsule composition, which may be desiccated for reconstitution, a scaffold and so on. The kit may contain suitable instructions.

Solutions of particular use can include those that have a viscosity that approximates that of naturally occurring synovial fluids. Viscosity measurements can be made using devices and methods as known in the art. Concentration of a polymer of interest can be adjusted to obtain a fluid having a viscosity that approximates that of the naturally occurring synovial fluid.

A composition of interest can comprise a monomer or polymer of interest or combinations of monomers and polymers of interest in a single solution. Thus, a synthetic synovial fluid can contain, for example, a polyGlcN and a GlcN-derivatized chondroitin sulfate. The specific amounts of each polymer can be adjusted at the design of the artisan and again the final amounts of each of the two polymers are configured such that the final solution approximates the viscosity of naturally occurring synovial fluid.

Once synthesized, the polymers are purified in a matter compatible with pharmaceutical administration practicing methods known in the art. The biological polymers of interest are then finished in a form suitable for storage and eventual use. Thus, the biological polymers can be suspended in a biologically compatible and pharmaceutically acceptable liquid diluent or can be desiccated or freeze-dried to form a dry powder for later reconstitution and administration. The solution can contain preservatives, buffers, osmotic agents and the like to obtain preparations with beneficial properties, such as extended shelf life, stability in solution and so on.

The liquid form is suitable for administration intra-articularly using known means, such as with a syringe and needle. Suitable amounts of replacement/supplemental fluid of interest are introduced into the joint as needed.

Suitable diluents include sterile water and biocompatible buffers such as phosphate buffered saline.

The products, devices and methods of interest are manufactured and packaged according to pharmaceutic standards. Thus, the products can be manufactured and assembled under, for example, good manufacturing practice standards as recognized by a respective regulatory agency, as known in the art. The products then can be packaged/kitted again following such good manufacturing practice standards.

The products and devices of interest can find use in any articular joint or with any bone or cartilage site in need of treatment. The composition and delivery means can be manipulated for the particular end use practicing known drug delivery materials and methods.

All references cited herein are incorporated by reference in entirety.

The invention hereinabove being described would be evident to one of ordinary skill in the art that various modifications and changes can be made to the teachings herein without departing from the spirit and scope of instant invention.

We claim:

1. A composition comprising a glucosamine, wherein said composition provides for a local concentration of about 2 mM glucosamine.

2. The composition of claim 1, wherein said glucosamine comprises a polymer.

3. The composition of claim 2, wherein said polymer comprises a biodegradable backbone.

4. The composition of claim 3, wherein said backbone comprises a naturally occurring polymer found in cartilage.

5. The composition of claim 4, wherein said naturally occurring polymer comprises chondroitin sulfate or hyaluronic acid.

6. The composition of claim 2, wherein said polymer comprises a polyglucosamine.

7. The composition of claim 1, comprising particles.

8. The composition of claim 1, comprising fibers.

9. The composition of claim 1, comprising a scaffold.

10. The composition of claim 1, further comprising a second biologically active ingredient.

11. The composition of claim 10, wherein said second active ingredient comprises a molecule found in cartilage, bone or extracellular matrix.

12. A parenteral composition comprising a glucosamine.

13. The parenteral composition of claim 12 further comprising a second active ingredient found in cartilage, bone or extracellular matrix.

14. The parenteral composition of claim 12 wherein said second active ingredient comprises a hyaluronic acid.

15. The parenteral composition of claim 12, wherein said second active ingredient comprises a chondroitin sulfate.

16. The parenteral composition of claim 12, wherein said glucosamine comprises a capsule or a fiber.

17. The parenteral composition of claim 12, wherein said glucosamine comprises a polymer.
